(19) United States
(12) Patent Application Publication
(10) Pub. No.: US 2006/0057618 A1
(43) Pub. Date: Mar. 16, 2006

(54) Determining data quality and/or segmental aneusomy using a computer system

(75) Inventors: James Richard Piper, Aberlady (GB); Ian Poole, Edinburgh (GB)

Correspondence Address: QUINE INTELLECTUAL PROPERTY LAW GROUP, P.C., P O BOX 458, ALAMEDA, CA, 94501, US

(73) Assignee: Abbott Molecular, Inc., a Corporation of the State of Delaware, Des Plaines, IL (US)

(21) Appl. No.: 11/208,018

(22) Filed: Aug. 18, 2005

Related U.S. Application Data
(60) Provisional application No. 60/603,218, filed on Aug. 18, 2004.

(51) Int. Cl. C12Q 1/68; G06F 19/00

(52) U.S. Cl. 435/6; 702/20

(57) ABSTRACT

A method and/or system for making determinations regarding samples from biologic sources including statistical methods for making meaning grouping of observed data and/or for determining an overall quality measure of an assay.

### CROSS REFERENCE TO RELATED APPLICATIONS

This application claims priority from provisional patent application 60/603,218, filed 18 Aug. 2004 and incorporated herein by reference.

This application is related to U.S. patent application Ser. No. 10,269,723 filed 11 Oct. 2002, which is a non-provisional of 60/378,760 filed 12 Oct. 2001, both of which are incorporated herein by reference.

U.S. patent application Ser. No. 10/342,804 filed 14 Jan. 2003 and its corresponding provisional patent application 60/349,318, filed 15 Jan. 2002 are incorporated herein by reference for all purposes.

### COPYRIGHT NOTICE

Pursuant to 37 C.F.R. 1.71(e), applicants note that a portion of this disclosure contains material that is subject to and for which is claimed copyright protection, such as, but not limited to, source code listings, screen shots, user interfaces, or user instructions, or any other aspects of this submission for which copyright protection is or may be available in any jurisdiction. The copyright owner has no objection to the facsimile reproduction by anyone of the patent document or patent disclosure, as it appears in the Patent and Trademark Office patent file or records. All other rights are reserved, and all other reproduction, distribution, creation of derivative works based on the contents, public display, and public performance of the application or any part thereof are prohibited by applicable copyright law.

### FIELD OF THE INVENTION

The present invention relates to the field biologic assays and data analysis. More specifically, the invention relates to a computer or other logic processor implemented or assisted method for making certain determinations regarding assays typically from biologic sources. In further embodiments, the invention involves systems, methods, or kits for performing screening and/or diagnostic tests for a variety of disease or conditions.

### BACKGROUND OF THE INVENTION

Normal human cells contain 46 chromosomes in 22 autosome pairs (often indicated using numbers 1 through 22) and 2 sex chromosomes (sometimes indicated as 23 and 24). Generally, normal cells contain two copies of every chromosome (other than the sex chromosome), Consequently normal cells also contain two copies of every gene, except again for genes lying on the sex chromosomes.

In congenital conditions such as Down syndrome and in acquired genetic diseases such as cancer, this normal pattern of two copies of every chromosome and two copies of each gene is often disrupted. Whole chromosome number can be altered, with cancer cells in particular showing patterns of gain or loss of whole chromosomes or chromosome arms. (The number of copies of a chromosome in a cell is also referred to as its “ploidy”.) In other cases, a chromosomal rearrangement may result in a portion of one or more chromosomes being present in more than or fewer than two copies. This portion can correspond to whole or parts of one or more genes. Thus, genetic abnormalities are often described in terms a gain or loss in copy number, where in different situations, copy number can refer to chromosomes, to genes, or more generally to contiguous sequences of DNA. Alterations in copy number may also be referred to as copy number imbalances.

Genes influence the biology of a cell via gene expression which refers to the production of the messenger RNA and thence the protein encoded by the gene. Gene copy number is a static property of a cell established when the cell is created; gene expression is a dynamic property of the cell that may be influenced both by the cell's genome and by external environmental influences such as temperature or therapeutic drugs.

In general, various patterns of copy number imbalance are characteristic of certain congenital abnormalities or certain cancers, and determination of the pattern of imbalance can inform diagnosis, prognosis and/or treatment regimes. Thus, it is frequently desired to measure and/or determine and/or estimate copy number imbalance in cells and/or tissues and/or material derived therefrom. Chromosomal imbalances are measured using a variety of techniques, such as quantitative PCR, in situ fluorescence measuring, and other techniques that attempt to count or estimate the number of specific genetic sequences. However, in many situations there is an increasing need for improved methods for detecting and/or measuring genetic imbalance.

The discussion of any work, publications, sales, or activity anywhere in this submission, including in any documents submitted with this application, shall not be taken as an admission by the inventors that any such work constitutes prior art. The discussion of any activity, work, or publication herein is not an admission that such activity, work, or publication was known in any particular jurisdiction.

### REFERENCES

A. D. Carothers, A likelihood-based approach to the estimation of relative DNA copy number by comparative genomic hybridization, Biometrics 53, 848-856, 1997.
J. Clark et al, Genome-wide screening for complete genetic loss in prostate cancer by comparative hybridization onto cDNA microarrays, Oncogene 22, 1247-1252, 2003.
J. Fridlyand et al, Statistical issues in the analysis of the array CGH data, Proc. Computational Systems Bioinformatics CSB'03, 2003.
J. Fridlyand et al, Hidden Markov models approach to the analysis of array CGH data. J. Multivariate Analysis 90, 132-153, 2004.
I. Miller and M. Miller, John E. Freund's Mathematical Statistics 6^{th} edition. Prentice Hall, 1999.
J. Piper et al, An objective method for detecting copy-number change in CGH microarray experiments, Proc. 3^{rd} Euroconference on Quantitative Molecular Cytogenetics, Rosenön, Stockholm, Sweden, 4-6 July 2002, pp. 109-114, 2002.
J. R. Pollack et al, Genome-wide analysis of DNA copy-number changes using cDNA microarrays. Nature Genet. 23, 4146, 1999.

### SUMMARY

The present invention involves techniques, methods, and/or systems useful for analyzing data typically related to biologic samples and most typically implemented on some type of logic execution system or module. Various aspects of the present invention may be incorporated into software for running a number of analysis on biologic detection or diagnostic systems, such as micro array diagnostic systems. While a number of specific diagnostic assays and details thereof are described below, some of which have independently novel aspects, the analysis methods of the invention have application to a variety of diagnostic and/or predictive situations in which data sets must be analyzed to determine relevant groupings and/or data quality.

In specific embodiments, the invention is directed to research and/or clinical applications where it is desired to assay or analyze samples containing biologically derived material, such as cellular material or nucleic acids. The invention according to specific embodiments is further directed to applications where it is desired to analyze sample assays by analyzing images of assay reactions, for example, images of one of various types of array chips for biologic detection or images of various cellular or tissue preparations suitable for imaging. In such a situation, the captured image data provides a digital representation of the observable data of the assay reaction. This image can be a two-dimensional image captured and analyzed within an information processing system, as will be understood in the art. According to embodiments of the invention, an image is digitally captured by and/or transmitted to an information processing system.

Specific embodiments are directed to techniques, methods and/or systems that allow automatic segmental aneusomy detection (SA) (this is referred to as segmental aneuploidy detection is some earlier work and prior applications) in microarrays, in specific examples in Comparative Genomic Hybridization (CGH) microarrays and analysis of related data sets.

Other specific embodiments are directed to techniques, methods and/or systems that allow automatic and objective determination of the quality of data sets such as those related to genomic microarray images. Quality is defined according to specific embodiments of the invention as described herein. In certain embodiments, the invention involves methods and/or systems for the prediction of data quality or an error rate of unknown samples by correlating that error rate to detectable features of the samples. In particular embodiments, Automatic Segmental Aneusomy Detection and/or Objective Data Quality determination can be used to accomplish or assist in diagnoses of a variety of diseases or other conditions.

The invention can also be embodied as a computer system and/or program able to analyze captured image data to estimate data quality and this system can optionally be integrated with other components for capturing and/or preparing and/or displaying sample data.

Various embodiments of the present invention provide methods and/or systems for diagnostic analysis that can be implemented on a general purpose or special purpose information handling system using a suitable programming language such as Java, C++, Cobol, C, Pascal, Fortran, PL1, LISP, assembly, etc., and any suitable data or formatting specifications, such as HTML, XML, dHTML, SQL, TIFF, JPEG, tab-delimited text, binary, etc. In the interest of clarity, not all features of an actual implementation are described in this specification. It will be understood that in the development of any such actual implementation (as in any software development project), numerous implementation-specific decisions must be made to achieve the developers' specific goals and subgoals, such as compliance with system-related and/or business-related constraints, which will vary from one implementation to another. Moreover, it will be appreciated that such a development effort might be complex and time-consuming, but would nevertheless be a routine undertaking of software engineering for those of ordinary skill having the benefit of this disclosure.

The invention and various specific aspects and embodiments will be better understood with reference to the following drawings and detailed descriptions. For purposes of clarity, this discussion refers to devices, methods, and concepts in terms of specific examples. However, the invention and aspects thereof may have applications to a variety of types of devices and systems.

Furthermore, it is well known in the art that logic systems and methods such as described herein can include a variety of different components and different functions in a modular fashion. Different embodiments of the invention can include different mixtures of elements and functions and may group various functions as parts of various elements. For purposes of clarity, the invention is described in terms of systems that include many different innovative components and innovative combinations of innovative components and known components. No inference should be taken to limit the invention to combinations containing all of the innovative components listed in any illustrative embodiment in this specification.

When used herein, “the invention” should be understood to indicate one or more specific embodiments of the invention. Many variations according to the invention will be understood from the teachings herein to those of skill in the art.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A-E illustrate an example of building an iterative model from multiple chromosome hybridization data to identify segments of sequences of detected genetic imbalance according to specific embodiments of the invention.

FIG. 2 is an example graph comparing sensitivity versus specificity of imbalance detection using methods according to specific embodiments of the invention compared to other methods.

FIG. 3 is an example of observed data captured as an array image with, for example, a reader either designed or modified for reading slides with different fluorescent labels.

FIG. 4 is an example graph comparing sensitivity versus specificity for isolated-target segmental aneusomy (SA) by “slope” and “basic” methods according to specific embodiments of the invention.

FIG. 5A-B are example scatter plots show the correlations with false positive rate (FPR) at alpha=0.01 (blue) and FNR at alpha=0.0001 (pink) of the features (A) slope and (B) the standard deviation of modal target ratios (“modal SD”).

FIG. 6 is an example scatter plot showing Eₚₒₛ (pink) and E_{neg} (blue) plotted against the same modal SD quality feature as illustrated in FIG. 5 above for FNR and FPR.

FIG. 7A-B are example scatter plots showing that Eₚₒₛ declines with (A) both increasing Geometric Mean Intensity and (B) increasing Geometric Mean Signal To Background Ratio (sig:BG), which could be a result of increased intensity.

FIG. 8 is an example scatter plot showing that the Median Adjacent Clone Ratio Difference behaves very similarly to modal distribution SD.

FIG. 9 is an example scatter plot showing that Eₚₒₛ declines as the variability of target clone intensity (CV) increases.

FIG. 10 is an example scatter plot showing that Eₚₒₛ is somewhat correlated with the proportion of saturated plus outlier pixels.

FIG. 11 is an example plot illustrating results of predicting objective Overall Quality Rating (OQR) by multiple regression according to specific embodiments of the invention.

FIG. 12A-B are two example plots illustrating the impact of the quality classes on SA performance where the data set has been triaged into three quality classes by the predicted value of OQR according to specific embodiments of the invention.

FIG. 13 is a block diagram showing a representative example logic and/or diagnostic system in which various aspects of the present invention may be embodied.

FIG. 14 (Table 2) illustrates an example of diseases, conditions, or statuses for which substances of interest can evaluated according to specific embodiments of the present invention.

### DESCRIPTION OF SPECIFIC EMBODIMENTS

Segmental Aneusomy Detection

Methods of the present invention can be most easily understood in the context of diagnostic assays that have some familiarity in the art. Use of the specific example herein of a particular microarray system should not be taken to limit the invention, which has applications in analogous data collection and analysis situations. In one known technique for detecting gene, chromosome, or DNA segment imbalance, a test sample of, e.g., whole-genome DNA that is to be analyzed is labeled with one fluorophore (e.g., Cy3) and hybridized to a microarray together with a similar quantity of a reference sample of DNA labeled with a different fluorophore, (e.g., Cy5) plus an excess of, for example, unlabeled competitor DNA (e.g., Cot1 DNA) to suppress hybridization signals from repeat sequence DNA.

Typically, the microarray is prepared with target sequence DNA areas or spots arranged in a systematic way. In one typical system, each spot of the micro array contains many copies of a known sequence of DNA, which are at times referred to as targets or target clones. In many systems, each target sequence will be represented by three replicate spots on the microarray. One known human whole-genome microarray contains 3 replicate spots containing many clones of each of 333 target DNA sequences. Typically, each target DNA sequence contains a well-defined portion of a DNA sequence from a single chromosome.

Thus, in a typical detection procedure using such a microarray, microarray target spots are hybridized with the test sample, reference sample and any other reagents and images are captured, showing Cy3 and Cy5 fluorescence at target spot areas. In this type of assay, the captured images represent the observable data from the assay. In example systems, captured images are typically corrected for artifacts such as background fluorescence, the spots segmented and identified, and the ratio of the test sample fluorescence to the reference sample fluorescence (e.g. Cy3 to Cy5) intensities is measured at each spot. Examples of such systems are described in the above referenced and incorporated patent applications. Following ratio normalization, the fluorescence ratios are expected to be about 1.0 for target spots with DNA sequences with corresponding (or genetically complementary) DNA sequences of which have the same copy number is the same in the test and reference samples, but different from 1.0 for spots for which the corresponding test DNA sequence copy number is in imbalance. An amplification or gain of copy number in the test sample will result in a larger ratio, while loss of copy number in the test sample will result in a lower ratio. In this discussion, the term ratio generally refers to normalized ratios.

A variety of statistical methods have been proposed or employed to determine whether the ratio for a particular target sequence averaged across its replicates is significantly different from 1.0. One such is the “p-value” method, as described in the coassigned patent application referenced above (U.S. patent application Ser. No. 10,269,723, Piper, filed Oct. 11, 2002). That method, in some specific embodiments, computes three values: (1) a significance level or p-value from the average ratio of the replicates for one target; (2) the variance among the target's replicate spot ratios; and (3) the variance of the ratios of other targets on the same microarray that are assumed or known or predicted to have balanced DNA copy number (such targets can also be referred to as “modal” targets.) The p-value method and some other statistical methods generally examine each target DNA sequence in isolation.

Example Segmental Aneusomy (SA) Detection

In a first aspect, the present invention involves systems and/or methods that detect imbalanced regions of a genome using microarray data from target spots from one or more target DNA sequences. Particularly in the case of constitutional genetic imbalances such as those associated with congenital abnormalities, but also in many cancer samples, it is common for a DNA sequence copy number imbalance to affect a contiguous region of the genome sequence, for example the gain of a whole chromosome 21 in Down syndrome, or the deletion of several megabasepairs of DNA in a microdeletion syndrome. The invention in specific embodiments uses co-occurrence of imbalance in one or more targets to increase the sensitivity and specificity of imbalance detection.

In particular embodiments, the invention analyzes the set of observed spot ratios by iteratively determining models of expected ratios that best explain the observed ratios. An expected ratio is the ratio that would be observed for a target from a given copy number in the test sample and another given copy number in the reference sample in a perfectly noise-free system that has optimum sensitivity and no signal attenuation. Since the copy number of the reference DNA is known, the unknown copy number of the test DNA can be determined from the expected ratio. A model according to specific embodiments of the invention groups target sequences into sequential sets of target sequences on the same chromosome that all have the same expected ratio. Herein, these sequential sets are referred to as segments. The base model is that all target ratios have a ratio value of 1.0 (also referred to as modal targets).

In building a model according to specific embodiments of the invention, each iteration adds one non-modal segment of one or more target sequences to the previous model. The non-modal (or positive) segment that is chosen is the one that causes the new model to best fit the data, using an optimization based on the statistical concept of likelihood. The new model is accepted if and only if the gain in log-likelihood is statistically significant. When only non-significant changes to the model are possible, it is regarded as complete.

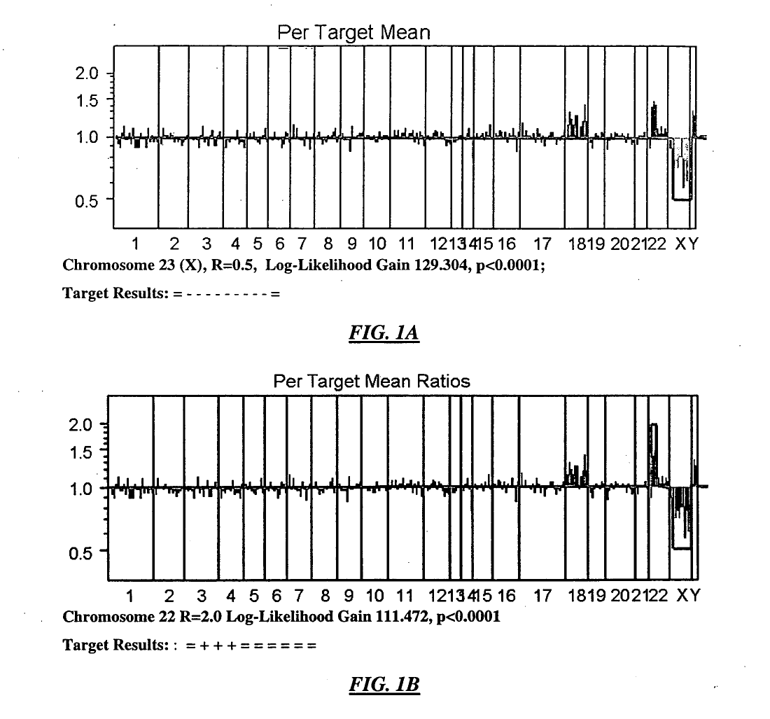

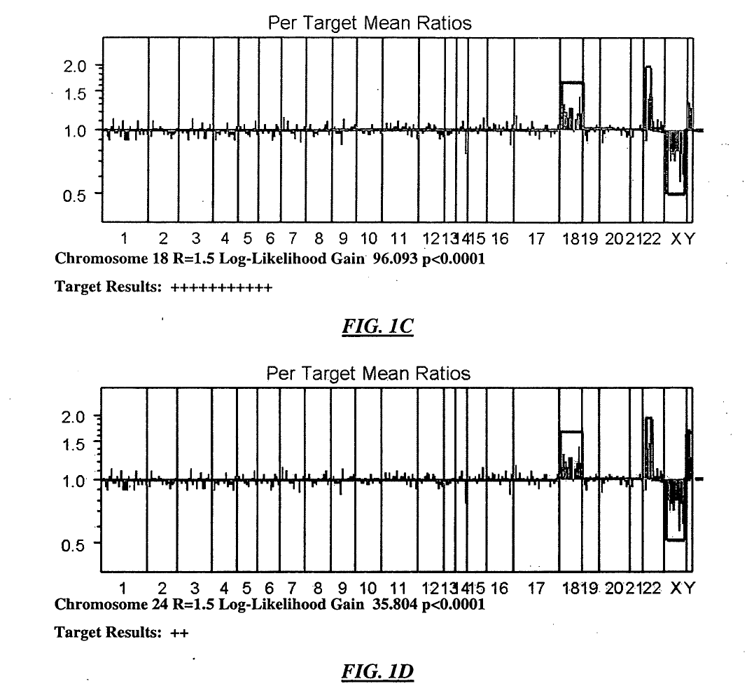

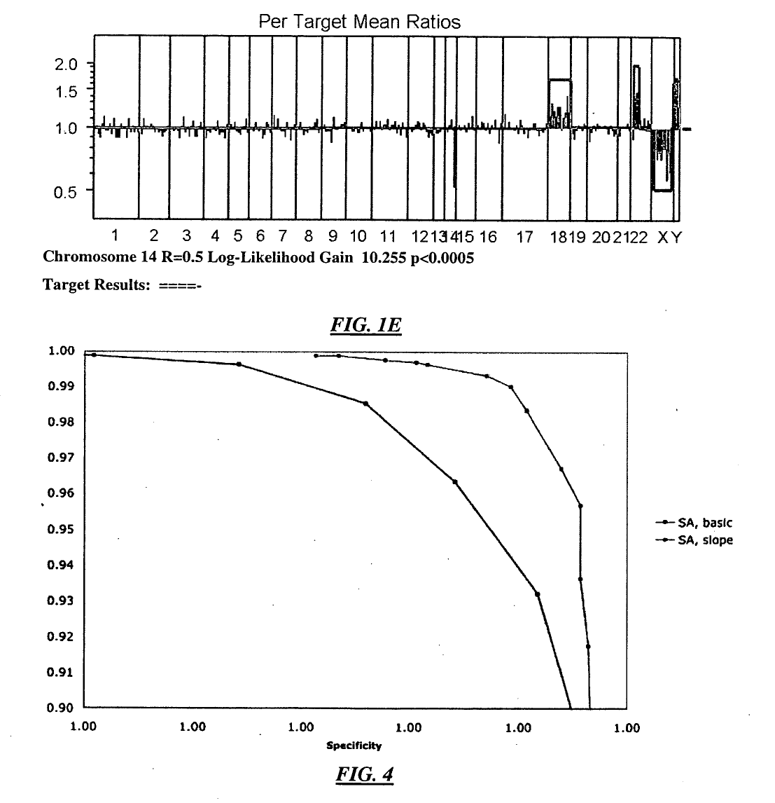

Model-building according to specific embodiments of the invention can be visually illustrated and conceptually understood by examination of FIG. 1A-E. While the process is straightforward to illustrate, for some applications of this method, such as for validated and repeatable diagnostics, it is desirable to have a mathematically deterministic and rigorous method of performing the data analysis, examples of which according to specific embodiments of the invention are described further below.

In the sequence shown, each successive model fits the observed data significantly better than the preceding model. In this example, the gain in log-likelihood at the 6th iteration had p>0.02 by the χ² test familiar in the art of statistical analysis and was therefore judged not significant; this caused the search for better-fitting models to terminate.

Segmental aneusomy detection according to specific embodiments of the invention has better performance than other methods if positive targets (i.e., those targets for which the corresponding test sample sequence has a DNA loss or gain) lie in segments of length two target sequences or more, and has at least equivalent performance in the detection of isolated positive targets.

Example Method

According to specific embodiments, the invention takes advantage of the fact that a test sample copy number change, whether involving a whole chromosome or part of a chromosome, usually will change the ratios at multiple sequential target spots. For purposes of this discussion, a contiguous set of DNA targets that all indicate the same copy number change in the test sample are referred to as a segmental change, or segment for short.

Methods of segment analysis have been considered in the context of applying cDNA clone expression microarrays to CGH analyses. The small sequence length of cDNA target clones results in very noisy ratio data when probed with whole-genome DNA, and the performance of individual targets is correspondingly poor. For example, Pollack et al (1999) described the use of “moving average windows” to detect single copy changes of sets of sequential cDNA target clones with 98% sensitivity and also 98% specificity, but did not apply any measure of significance to the detected segments. Clark et al (2003) proposed the use of Lowess curve fitting to the sequence of all target clone ratio data to detect possible segments with altered ratio, followed by the Mann-Whitney U test to provide a significance level for a candidate segment. One application of a segment technique to BAC/PAC clone microarrays specifically manufactured for CGH analysis was described by Fridlyand et al (2003, 2004), who fitted hidden Markov models (HMM) to the sequence of target ratios from array CGH analysis of cancer cell lines.

As Clark et al (2003) discussed, segment identification has two components. First, one or more candidate segments must be proposed. In some embodiments of the current invention an exhaustive search proposing all possible segments is used. This neatly avoids the issue of positive segments possibly being missed by the candidate generation method, and the invention can employ methods to make the subsequent computations very efficient. Second, a measure of the value or significance of each candidate segment is used in order to choose good segments but reject less good segments, and thereby discriminate true copy number changes from the effects of random noise.

Aspects of the present invention can be further understood with reference to a metaphase cell CGH analysis method described by Carothers (1997), who proposed a maximum-likelihood framework for iteratively building a model of a CGH chromosome ratio profile as a series of contiguous segments of profile points. In Carother's model, every point in a given segment had the same test and reference copy numbers. Model construction was constrained to be consistent with the “crosstalk” between neighboring points on the chromosome profile, and employed a principle of parsimony, that the model was only allowed to become more complex if the resulting likelihood increase was significant according to an appropriate statistical test.

Specific embodiments of the present invention make use of one or more of: a likelihood framework, an iterative method, a parsimony principle, constraints, and the specification of the model in terms of underlying “expected ratios” derived from test and reference copy numbers. Crosstalk is generally not present on microarrays, and its role as a constraint on the solution has been replaced by (i) insistence that segments with non-modal expected ratios comprise sequential genomically-ordered target clones on the same chromosome, (ii) theory-based constraints on the allowable values of the expected ratios.

One specific example of the likelihood function to be maximized can be understood as follows. (1) Let the genomically-ordered set of targets on the microarray be indexed by i, i=1 . . . k, and replicate spots within one target be indexed by r, r=1 . . . nᵢ. Typically nᵢ=3 for all i, and typically i has values such as 333 or 287 depending on the number of targets provided or analyzed on a particular microarray. Let the observed ratio data for a spot r belonging to target i be designated as yᵣᵢ, comprising an underlying value (constant across replicates for a target Yᵢ) plus an error term eᵢᵣ such that yᵣᵢ=Yᵢ+eᵢᵣ and the observed mean ratio across the replicate spots of target i is designated yᵢ and the set of observed ratios for the set of targets on the microarray is denoted y. (While log-ratios could be used, with only a slightly different theoretical development, in practice in tested situations, the log-ratio formulation did not perform as well as when using the ratios themselves.)

A model according to specific embodiments of the invention is a set of “expected ratios” denoted cᵢ representative of an underlying hypothesis about the test and reference copy numbers at each target locus. The set of expected ratios for the complete set of targets on the microarray is denoted c.

To choose the best fitting model by maximum likelihood, the invention maximizes the log-likelihood of y given c: L(c)=log(p(y|c))

Assume the target ratios are statistically independent of each other, specifically: p(yᵢ|c)=p(yᵢ|cᵢ) and p(yᵢ|cᵢ)=p(yᵢ|cᵢ,yⱼ),i≠j. This allows us to write: L(c)=log(p(y|c))=Σᵢp(yᵢ|cᵢ), the summation being taken across all targets i. Assuming normal distributions, L(c) can be computed from the formula: L(c)=a−Σᵢ(yᵢ−cᵢ)2vᵢ, where a is a constant, and vᵢ is the variance of yᵢ.

The variance vᵢ can be modeled as uᵢ+w, where uᵢ=within-target-variance/nᵢ (typically 3), and w is the “target noise” (variance among the set of targets of the target mean ratios when normal copy number test and reference DNAs are hybridized at all target loci). Assuming that segment transitions are comparatively rare, w can be estimated approximately from the set all uᵢ and the variance of the distribution of adjacent target differences (yᵢ−yᵢ₋₁) as follows: for given i, var(yᵢ−yᵢ)=var(yᵢ)+var(yᵢ₋₁)=vᵢ+vᵢ₋₁, where var(.) is the variance of a random variable; this is a well-known theorem. Though vᵢ and vᵢ₋₁ may not be the same as each other, considering average values along the entire set of targets (e.g., the entire genome), then E(var(yᵢ−yᵢ₋₁))=2E(vᵢ), where E(.) is the expected value of a random variable across the set indexed by i. Substituting vᵢ by uᵢ+w, noting that E(w)=w because w is a constant of the chromosome (or chip) rather than a target-dependent variable, and rearranging, results in w=0.5 E(var(yᵢ−yᵢ₋₁))−E(uᵢ).

Both E(var(yᵢ−yᵢ₋₁)) and E(uᵢ) can be estimated from the data. E(var(yᵢ−yᵢ₋₁)) is approximated by the variance of the set of all adjacent target ratio differences (yᵢ−yᵢ₋₁), denoted var{(yᵢ−yᵢ₋₁)}. When estimating var{(yᵢ−yᵢ₋₁)}, exclude the differences across segmental ratio changes, which of course are initially not known. This is achieved in specific embodiments by rejecting outlier differences, based on thresholds established from the first and third quartiles±three times the interquartile range. Similarly, when computing the average within-target variance E(uᵢ), outlier variances are discarded.

Now maximize the likelihood L(c) over the set of possible values of c (expected target ratios), under constraints appropriate to the diagnostic analysis being performed.

A model employed in preferred embodiments of the present invention has no smoothness term (targets are statistically independent, and actual target ratio data when plotted against target sequence number always looks “jagged”), but if there were no constraints at all then it is possible than the optimal solution would be the expected ratio values simple equal the observed values (e.g., c=y).

In an example embodiment, two constraints appropriate to particular CGH microarray diagnostic applications are used. First, all expected ratios cᵢ must either be 1.0, or must deviate from 1.0 by an amount that fits a model that the test and reference DNAs have copy numbers of 1, 2 or 3 everywhere. (While this constraint is particular appropriate for congenital imbalances, other copy numbers may be more appropriate for detection of other cellular imbalances, such as those due to cancer, retroviral infection, or other conditions)

Note that the Y chromosome targets are not treated as having copy number zero in a female sample due to the high degree of homology between these targets and the X chromosome and/or autosome sequences. Instead, Y is assumed to have copy number of 0.5 in a female sample, leading to theoretically expected ratios of 0.5 in female test sample vs. male reference sample, 2.0 in male test sample vs. female reference sample, and 1.0 in sex-matched test and reference sample hybridizations. While this treatment of Y is a simplification, it has been found to work fairly well in practice, as has ignoring homologies other than between Y and X among targets.

In specific embodiments of the method, these constraints are applied by requiring that cᵢ=1+s(Rᵢ−1) where Rᵢ=tᵢ/rᵢ is one of {0.5, 1.0, 1.5, 2.0}, and s is a constant of the chip that will end up being estimated from the data. The s value in this discussion can be understood to represent the attenuation of a measured non-modal ratio as compared with the expected ratio value. This value is sometimes referred as a “slope” value as a result of some analogies to earlier work wherein measured ratio was plotted against expected ratio for a single experiment where there are different expected ratios, resulting in straight line with slope s. As a second constraint, while in principle, 0<s<1, to preclude trivial solutions, constrain s such that 0.25<s<1.0.

In further specific embodiments, the search proceeds by hypothesizing constrained changes to the expected ratios in the ordered sequence of targets. In each iteration, add whichever single non-modal segment (or new modal-ratio segment placed in the interior of an existing non-modal segment, e.g. in chromosome X) maximizes the likelihood L(c), by searching through a space defined by the following 4 free parameters:
1. L_{b}, the index of the first altered target.
2. Lₑ, the index of the last altered target. The search is limited to segments contained within a single chromosome.
3. q, the expected “ratio deviation” (i.e., from 1.0) of the altered targets assuming that slope=1. In specific embodiments, q is drawn from the set of 4 distinct allowed values expressed as (t/r−1), see above. Note that c=1+sq.
4. s, the current best estimate of slope for this chip.

The difference in the log-likelihood between the current and previous models, when multiplied by 2, is χ² distributed with degrees of freedom equal to the number of additional parameters added to the model (Miller and Miller, 1999, p. 404). Each iteration of model building is therefore evaluated by comparing twice the log-likelihood difference between current and previous models with the χ² distribution with 4 degrees of freedom. If the log-likelihood gain falls below the critical value for a chosen significance threshold, the search terminates. In other words, over-fitting of the model is avoided by use of a formal significance test.

In further specific embodiments, note that although the optimization may be done on a per-chromosome basis, slope s and target ratio variance w also have chip-wide components. Therefore, in specific embodiments, it is appropriate to search across the entire set of targets on the chip simultaneously, while not allowing potential segments to extend beyond the ends of the individual chromosome. The final result is a description of copy number changes for the entire chip.

The search space is relatively well-constrained. L_{b} and Lₑ must lie on the same chromosome; this limits the possible number of segment end-point pairs in one example chip to in the order of 2000; q can take only 4 possible values. As noted above, s is constrained to lie in the range 0.25<s<1.0. Brute-force search for optimal s with an increment in s of, say, 0.01 would not be too arduous and can be employed in specific embodiments. However, a preferred method is to note that L(c)=a−Σᵢ(yᵢ−cᵢ)²/vᵢ can be expressed as a function of s, as follows:

$$\begin{matrix}L ( c ) = a - {\sum }_{i} {( {y}_{i} - {c}_{i} )}^{2} / {v}_{i} \\ = a - {\sum }_{i} ( {y}_{i}^{2} - 2 {y}_{i} {c}_{i} + {c}_{i}^{2} ) / {v}_{i} \\ = a - {\sum }_{i} ( {y}_{i}^{2} - 2 {y}_{i} ( 1 + {sq}_{i} ) + {( 1 + {sq}_{i} )}^{2} ) / {v}_{i}\end{matrix} \qquad ( eqn 1 )$$

Given particular values of q, Lb and Le at some given point in the search, the value of s which maximises L(c) at those values can be found by differentiating the final expression above, and finding where the derivative is zero:
dL(c)/ds=−Σᵢ(−2yᵢqᵢ+2qⱼ+2sqᵢ²)vᵢ, which is zero when
s=(Σᵢqᵢ(yᵢ−1)/vᵢ)/(Σᵢqᵢ²/vᵢ) (eqn 2)
If the optimum value of s lies outside the allowed range 0.25<s<1.0, then the triple {q, L_{b}, Lₑ}is eliminated from further consideration.

In further specific embodiments, equation 1 also provides a basis for efficient computation of L(c) in the subsequent iteration. Since at any one point in the search the current hypothetical next segment change is limited to a single chromosome, the value of L(c) contributed by each other chromosome is of the form Lⱼ(cⱼ)=Aⱼ+Bⱼs+Cⱼs², where j indexes the chromosome, cⱼ is the subset of c belonging to chromosome j, and Aⱼ, Bⱼ and Cⱼ are constants. The sums below are taken over all targets i belonging to chromosome j (symbolically, iδj):
Aⱼ=Σ_{iδj}(yᵢ−1)²/v₁
Bⱼ=−2Σ_{iδj}qᵢ(yᵢ−1)/vᵢ
Cⱼ=Σ_{iδj}qᵢ²/vᵢ

The terms Aⱼ are in any case constant throughout the analysis. While searching for a new segment in chromosome k, the invention can pre-compute the terms Σ_{j≠k}Bⱼ and Σ_{j≠k}Cⱼ, which immediately provide the contribution of the remaining 23 chromosomes to L(c) and its derivative with respect to s. With these optimizations, the entire SA method becomes usable in practice, for example requiring just one or two seconds to compute to completion on a 667 Mhz PowerPC G4.

As an alternative to the method described above, instead of the value of slope s being re-estimated at each iteration of the algorithm as has been described, a segmental aneusomy detection algorithm can be implemented as follows.
1. Find the segment with the highest likelihood of being non-modal and compute the average of the observed ratios of the targets in the segment. Iterate this process until all segments whose likelihood gains are significant by the chi-square test have been found.
2. Find the best fit of the set of average observed segment ratios to the set of expected ratios. This step will estimate a value for the slope parameter s. The fitting must be constrained to plausible values of s.
3. Merge adjacent segments that have the same expected ratio. Segments detected at the first step which are allocated an expected ratio of 1.0 may indicate that the sample contains a mixed population of genomic clones (a “mosaic” sample). They should therefore not be discarded, and instead should be presented as anomalous to the user.
   Experimental Results

In one set of experimental investigations, 515 microarray images were collected from experiments with microarrays containing either 287 targets or 333 targets, each with 3 replicate spots. The test DNAs used in these samples were mostly from various cell-lines which had either a known whole chromosome gain or a known microdeletion; a minority of samples used normal test DNA. 8 target clones previously identified as consistently (i.e., not randomly) and commonly being the cause of false positive or false negative detection events were excluded from the analysis of all samples using the microarrays that contained 287 targets; in the samples that used the microarrays with 333 targets, all target clones were included in the analysis.

Performance was evaluated in terms of the false negative rate (FNR) and false positive rate (FPR) on a target by target basis. FNR═FN/GTP, i.e., the number of false negative targets divided by the number of ground-truth positive targets. Missing targets were excluded from both numerator and denominator. Similarly FPR═FP/GTN. Results are mostly reported here in terms of analytical sensitivity (1-FNR) and analytical specificity (1-FPR).

In order to generate receiver operating characteristic (ROC; i.e., sensitivity vs. specificity) data, analyses were repeated with a wide range of χ² probability thresholds.

Because the available data sets consisted mostly of hybridizations by trisomy cell-lines, with relatively few examples of microdeletions, microduplications or other small imbalances, the target mean ratio data were analyzed in four different ways in order to simulate the issues that would be posed by small segments and isolated target copy number changes.

In one analysis, the SA method as described was applied to the set of target clone data in its original genomic order. This is referred to below as “standard SA”. In all microarrays with 287 targets, chromosome Y provided an example of a segment of length 2, and in a substantial number of samples the DiGeorge Syndrome deletion region of chromosome 22 was an example of a segment of length 3. All other non-modal segments had length 7 or more.

In a second analysis, the order of the target clones was permuted or “shuffled” into a reordering intended to separate at least some of the clones in long non-modal segments into segments of 1, 2, 3 or 4 adjacent clones. The permutation was semi-random so that a different reordering was used for each sample. The X and Y chromosomes were left unshuffled. The SA method as described was then applied to the set of target clone data in shuffled order. Sex chromosome targets were analyzed in the standard fashion, with segments allowed to be of any length, so that the slope estimation could “get off to a good start”. This is referred to below as “shuffled SA”.

In a third analysis, as a temporary measure for this simulation experiment only, the SA algorithm was additionally constrained so that the only possible candidate segments on autosomes consisted of single target clones. Thus every autosome target was potentially detectable as an isolated target only. This simulation provided a very large set of isolated targets, much larger than could be envisaged if real data had to be provided for this purpose. This is referred to as “isolated target SA”.

For comparison, the original p-value method (PV; for a full description, see Piper, 2002) was also applied, with FN counting restricted to the autosome ground truth positive targets only so that a direct comparison could be made with the isolated target method above.

In each case, FPR was based on all targets (i.e., including the sex chromosomes). FPR for isolated target SA was as generated by standard SA, because this generates more FPs than isolated target SA.

In order to get a clearer idea of the influence of segment length on performance, a two dimensional histogram of the number of target clones detected vs. the true length of a segment was extracted from the “shuffled SA” analysis. A single suitable value of the χ² probability threshold was used.

The constrained segmental aneusomy (SA) method described above is referred to as the “slope” method. There is a simpler alternative, which we refer to as the “basic” method. In the basic method, the ratio chosen to model any potential segment of observed ratio data is just the mean observed ratio across all the targets in the segment. In other words, this model has neither the notions of “allowed expected ratios” nor of “slope”. Preliminary experiments showed a high likelihood of false-positive segments containing just a few targets which randomly all had a small non-modal ratio “going in the same direction”, so a single ad hoc constraint proved to be necessary: that a segment's model ratio must be either <0.85 or >1.15.

Results and Discussion

FIG. 2 is an example graph comparing sensitivity versus specificity of imbalance detection using methods according to specific embodiments of the invention compared to other methods. FIG. 2 compares sensitivity versus specificity (also referred to as ROC) curves from the four methods: standard SA and shuffled SA on all targets, and isolated target SA and PV for autosome targets only. These results show clearly that SA performs better than PV; the improvement is dramatic if the copy number change involves segments of length two or more target clones. But the improvement is also substantial when SA is artificially limited to segments of length one target clone.

Table 1 illustrates the two-dimensional histogram of counts of non-modal segments present in the data analyzed by SA following target order “shuffling”, when the χ² threshold was chosen to give about one false positive per 3 microarrays. The histogram is indexed by a segment's true Length in the vertical direction, and by the number of target clones from the segment that were actually Detected in the horizontal direction. The results show that segment detection performance is excellent for segments with three or more target clones.

TABLE 1

| | D | | | | | | | | | | | | | | |
|---|---|---|---|---|---|---|---|---|---|---|---|---|---|---|---|
| | 0 | −1 | −2 | −3 | −4 | −5 | −6 | −7 | −8 | −9 | −10 | −11 | −12 | −13 | −14 |
| L1: | 586 | 1002 | | | | | | | | | | | | | |
| L2: | 156 | 25 | 1233 | | | | | | | | | | | | |
| L3: | 23 | 1 | 23 | 435 | | | | | | | | | | | |
| L4: | 1 | 2 | 7 | 16 | 175 | | | | | | | | | | |
| L5: | 0 | 0 | 0 | 2 | 6 | 99 | | | | | | | | | |
| L6: | 0 | 0 | 0 | 0 | 0 | 1 | 53 | | | | | | | | |
| L7: | 0 | 0 | 0 | 1 | 0 | 0 | 14 | 127 | | | | | | | |
| L8: | 1 | 0 | 0 | 0 | 0 | 0 | 0 | 1 | 74 | | | | | | |
| L9: | 1 | 0 | 0 | 1 | 0 | 1 | 0 | 1 | 29 | 414 | | | | | |
| L10: | 0 | 0 | 0 | 0 | 0 | 0 | 0 | 0 | 0 | 0 | 1 | | | | |
| L11: | 0 | 0 | 0 | 0 | 0 | 0 | 0 | 0 | 0 | 0 | 0 | 0 | | | |
| L12: | 0 | 0 | 0 | 0 | 0 | 0 | 0 | 0 | 0 | 0 | 0 | 0 | 0 | | |
| L13: | 0 | 0 | 0 | 0 | 0 | 0 | 0 | 0 | 0 | 0 | 1 | 0 | 7 | 20 | |
| L14: | 0 | 0 | 0 | 0 | 0 | 0 | 0 | 0 | 0 | 0 | 0 | 0 | 0 | 29 | 90 |

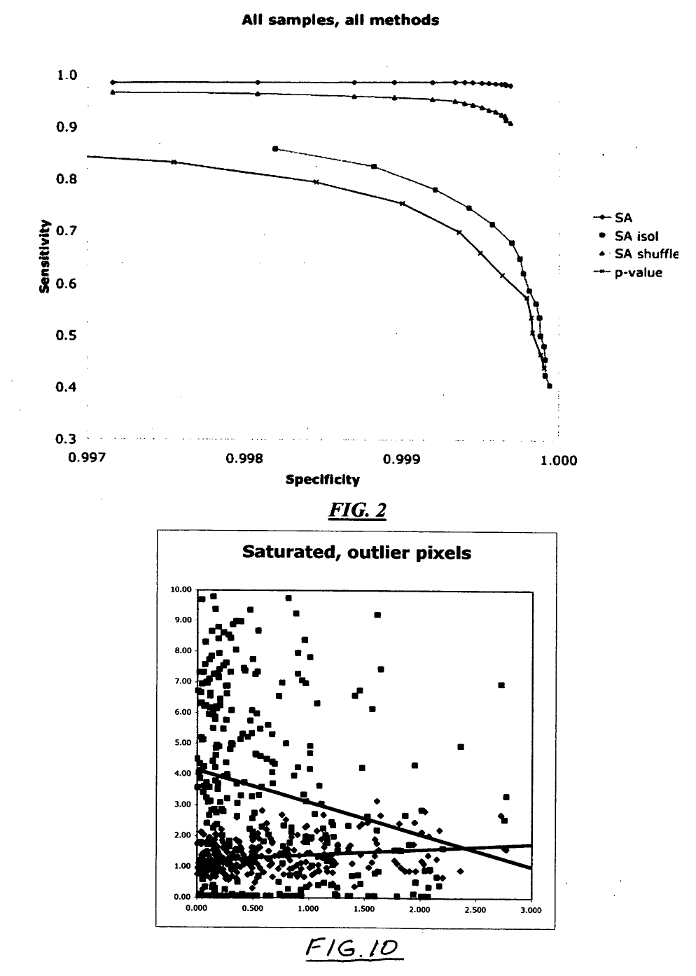

FIG. 4 shows ROC curves for isolated-target SA by the “slope” and “basic” methods, measured on a 110-chip subset of the data. The “slope” SA method outperforms the “basic” method in the detection of isolated target clones. This is believed to be chiefly due to the following. In order to be detected, a segment's log-ratio multiplied by the slope must be at least 50% of the smallest allowed model log-ratio. In other words, the method imposes a minimum ratio condition on the isolated clones. The minimum ratio is dependent on the slope and is therefore specific to each sample. Because of this, it eliminates false positives more efficiently than does the overall ratio threshold used by the “basic” method. The “basic” method does nevertheless have some advantages. Most notably, it will likely detect mosaic copy number changes rather better than the slope model.

Example Application to Pre and Post-Natal Genetic Testing

In further embodiments, the invention can be used with array comparative genomic hybridization (aCGH) in clinical and/or research settings to detect segmental and whole chromosome changes in copy number. A particular specific example uses a Tecan HS4800 Hybridization Station in combination with the GenoSensor™ Reader. In one example embodiment, hybridizations are performed on an array containing 333 clones spotted in triplicate. In a preferred array, all telomeres and regions associated with known microdeletions/microduplications of interest are represented by two or more closely spaced target sequences on the array, with target specificity determined by analysis such as PCR or FISH against normal peripheral blood specimens (PBS) to avoid polymorphic targets.

According to specific embodiments of the invention, a user software package (e.g., the GenoSensor software) uses statistical analysis methods of segmental aneusomy (SA) as described herein to improve sensitivity and specificity. In further embodiments, an overall quality of hybridization indicator as described below can also be employed.

In experimental tests, this new array and assay format significantly reduces time to results detecting congenital genetic imbalances (e.g., pre-natal, post-natal, and pre-implantation) while improving assay performance. For example, time to results starting with purified DNA in one assay has been reduced from 96 hours to 36 hours while the coefficients of variation and reproducibility have improved. Further optimizations are expected to reduce the turn around time even further.

Thus, in specific embodiments, a diagnostic system and/or method according to the invention can be optimized to detect chromosomal imbalances that are a common cause of developmental disorders such as mental retardation/developmental delay, physical birth defects and dysmorphic features. Currently, metaphase karyotype analysis is the gold standard in postnatal diagnostics of chromosome aneusomies, while fluorescence in situ hybridization (FISH) with probe(s) targeting submicroscopic genomic region(s) is the gold standard for detection of microdeletion and microduplication syndromes. The present invention in specific embodiments involves using comparative genomic hybridization (CGH) to in one assay diagnose chromosome aneusomies and microdeletion and microduplication syndromes. In specific embodiments, a detection system or method according the invention can be optimized for prenatal, postnatal, or embryonic pre-implantation diagnostic of these DNA sequence imbalances. Thus, in specific embodiments, the invention uses (Array-CHG) aCGH, (the application of CGH technology to chromosomal clones bound to a solid support) where each target clone is well-characterized and mapped to a specific chromosome region. An aCGH analysis according to specific embodiments of the invention allows highly sensitive detection of unbalanced genomic aberrations and can provide for the diagnostic detection of whole chromosome aneusomies, microdeletions, microduplications and unbalanced subtelomeric (subTel) rearrangements in a single assay.

The SA method of the invention can be used to enable a highly reproducible, automated aCGH assay format that does not require reciprocal hybridizations, and reliably detects copy number abnormalities (CNAs) from both fresh and fixed peripheral blood (PB) or cell line specimens.

Automated Platform

In preferred embodiments, the analysis methods of the invention can be incorporated into a CGH platform that automates hybridization and washing, automates image capture and data analysis, assesses the quality of the assay, and reports qualitative results (gain, loss, no change). The following modifications can be used to enable some example current systems to perform according to the invention: a) modified microarray labeling/hybridization kit, b) extended-content microarrays on glass slides, c) Tecan HS4800 hybridization station running proprietary hybridization protocol, and d) GenoSensor slide reader with software algorithms including the methods described herein.

aCGH Arrays and Target Sequence (Clone) Selection

A CGH array that was developed to perform specific assays of interest using methods of the invention consists of 333 genomic target DNA sequences (or clones). For clone selection, regions of interest were identified through publications, collaborators and national genetics meetings. At a minimum 3 clones were chosen per chromosome arm (6 per chromosome), for increased confidence in detecting gains/losses of a whole chromosome or chromosomal segments. The array contains 82 subtelomeric clones and 29 clones in known microdeletion/microduplication regions. Each telomere is represented by two clones, except for the acrocentric chromosome p arms. Each microdeletion/microduplication region is covered by 2-5 clones. The identity of each clone was confirmed by PCR assays with clone specific primers, and the specificity and cytogenetic location of each clone was verified by FISH.

For an example aCGH assay, test and normal reference DNA samples are random-prime labeled with Cyanine 3-dCTP, and Cyanine 5-dCTP (Perkin Elmer). Following additional purification, test and reference probes are combined in the aCGH hybridization buffer and hybridized to the 333-clone array on a Tecan HS4800 hybridization station for 24 hours, followed by automated wash and scanning of arrays.

Image and Data Analysis Software

In an example system, array images are captured with a reader modified for reading slides. Software associated with the reader controls image acquisition, analysis, and data reporting. The software identifies spots based on the DAPI signal, measures mean intensities from the green and red image planes, subtracts background, determines the ratio of green/red signal, and calculates the ratio most representative of the modal DNA copy number of the sample DNA. For each target, the normalized ratio, relative to the modal DNA copy number, is then calculated and the significance of the individual change reported. FIG. 3 is an example of observed data captured as an array image with, for example, a reader either designed or modified for reading slides with different fluorescent labels.

Using segmental aneusomy analysis as described above allows for highly-sensitive detection of segmental CNAs. In addition, the software can include predictive quality control features, including a quantitative rating of overall assay and image quality (Quality Measure) as described below, and can also include such things as a measure of the completeness of spot segmentation and the reliability of spot identification, and image focus.

Thus, the new data analysis and quality rejection algorithms allow for a) rejection of poor quality data based on the experimentally selected cutoff for the Quality Measure parameter, and b) choosing the appropriate level of probability to count changes in genomic copy numbers as “real.”

### Objective Assessment of Quality

According to further specific embodiments, the current invention involves one or more methods and/or systems providing a general framework for an objective definition of genomic microarray analysis quality, specific definitions of “quality measures”, and a methodology for automatically estimating quality measures from measurable “quality features”. In specific embodiments, parameters of an estimation can be trained by example chip images for which the true copy numbers target sequences are known (e.g., known samples).

Results that demonstrate the feasibility of this approach in the context of the segmental aneusomy (SA) method for detecting copy number change are presented below. The invention has a variety of applications, including in vitro diagnostic (IVD) microarray analysis software.

Introduction

The ability of a microarray experiment to correctly detect genomic copy number changes is related to at least two factors. Firstly, the ratio measured for a hybridized target where there is a copy number change must be sufficiently different from the ratios of hybridized targets with the usual or modal copy numbers. Secondly, random fluctuations in measured ratio values must be sufficiently low. Alternatively expressed, there must be sufficient signal to distinguish positive events from the noise inherent in the negative events. Various measures of signal are possible, for example the ratio change on positive control target clones, or the value of the slope that relates observed to expected ratios such as is returned by the Segmental Aneusomy procedure already described. Various measures of noise are also known in the art, for example the standard deviation of ratio changes on negative control target clones, the coefficient of variation among replicate spots of a target, the correlation of the test and reference intensities of individual pixel values within a spot, or the ratio of average signal to average background. Experienced users of microarrays sometimes make use of these measures in an ad hoc fashion to grade the quality of a microarray experiment.

In N. P. Carter, H. Fiegler, and J. Piper (2002) “Comparative Analysis of Comparative Genomic Hybridization Microarray Technologies: Report of a Workshop Sponsored by the Wellcome Trust”, Cytometry 49:43-48, it was proposed that the quality of control experiments (where positive and/or negative hybridized targets are known) can be measured by dividing the slope of observed to expected ratio by a composite measure of ratio noise. This combined individual measures of signal and noise into a single, more powerful, quality measure but did not explain how to use any such measurements from the image to estimate the quality of a microarray analysis applied to an unknown sample.

Specific embodiments of the present invention provide one or more of the following advantages: firstly, replacing ad hoc representations of quality outcome by an objective measure that directly predicts the likelihood of experiencing errors in the detection of hybridized targets that are positive or negative for copy number change but whose status is not known a priori; and secondly, optimally incorporating measures of signal and noise, such as those mentioned, together with measurements of other aspects of quality, to form a single objective measure.

Defining Quality

There are at least two alternative approaches familiar in the art for defining quality. The first is to ask one or more experts how they judge each particular microarray image. It can be expected that the answer may be based both on what the chip image looks like, for example to a human viewer, and on values provided by analysis software, for example exposure times, signal to background ratios, and so on. Given enough examples and enough expertise, this approach can be developed into a formal and semi-quantitative system, as some previous work may have demonstrated.

However, in specific embodiments, the invention provides a more detailed look at the underlying purpose of quality measurement. According to specific embodiments, the current invention adopts the view that a quality measurement system should be able to predict the likely failure rates of a microarray experiment. In other words, in an actual application of the array system to a new sample, there is an underlying genomic ground truth, that is generally unknown. There is also an analysis result, which is generally known. There may be errors in the analysis result compared with the genomic ground truth, with a corresponding “true” false positive (FP) and false negative (FN) rate, but generally one cannot “know” any of these from the results of the analysis.

According to specific embodiments of the invention, a quality measurement method and/or system is used to predict the true FP and FN rates (or some related value). Ideally, the estimate will be close to the unknowable true FP and FN values. In short, a quality measure according to specific embodiments of the invention predicts an error function. Given enough experience and expertise, previous semi-quantitative approaches might also be made to do this, but they would always to some extent be subjective. Thus, the present invention proposes a more fully objective measure.

Quality Outcomes: FNR, FPR, and NIR

In the case of CGH microarray experiments looking for DNA copy number change, there are generally three types of failure: false negative targets, false positive targets, and non-informative targets (e.g. those with too few acceptable replicate spots). In controlled experiments, generally the ground truth for each target can be known, and so in these experiments one can measure the false negative rate (FNR), the false positive rate (FPR), and the proportion or rate of non-informative targets (NIR).

According to various specific embodiments of the invention, any suitable combination of these three measurements could provide a fully objective definition of chip quality. But note that while FPR and FNR are in principle unknown in a novel experiment, and so must generally be predicted from other data, NIR is directly available from the results of existing software analysis. Thus, in specific embodiments, the invention can retain NIR as a completely separate quality measure. For this reason, the present invention in specific applications defines chip quality as discussed below by a weighted sum of FNR and FPR or their analogs.

Quality Features

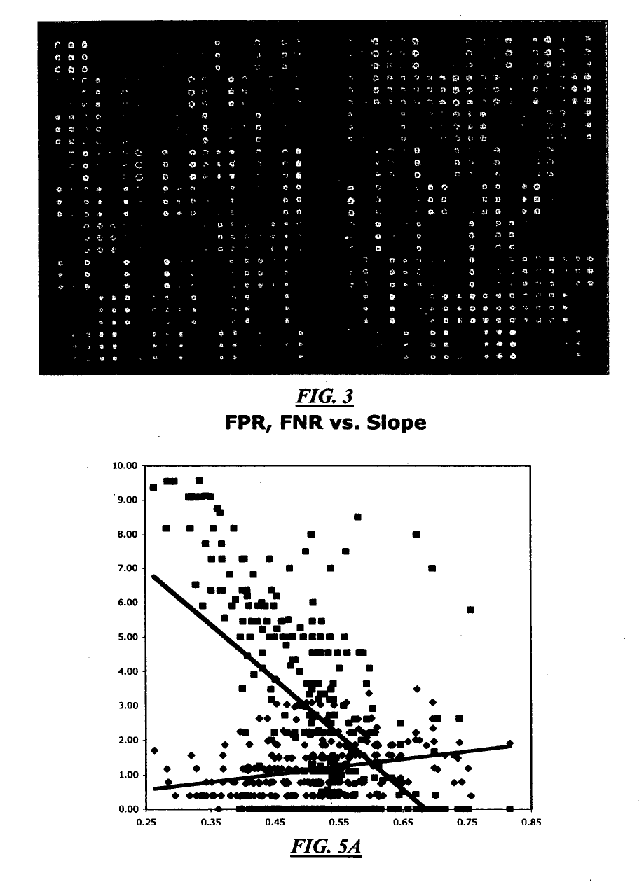

During the analysis of a microarray image, a number of features that relate to the quality of the microarray become available. Examples are (1) the variance of target ratios, (2) the slope or attenuation of observed to expected ratio, both of which are generated by the Segmental Aneusomy algorithm described above. In effect the first is a measure of microarray noise, while the second is a measure of ratio signal. Unsurprisingly, error rates measured in control experiments show considerable correlation with these features. FIG. 5A-B are example scatter plots show the correlations with false positive rate (FPR) at alpha=0.01 (blue) and FNR at alpha=0.0001 (pink) of the features (A) slope and (B) the standard deviation of modal target ratios (“modal SD”).

There is a clear relationship between FNR and slope: as slope increases, FNR drops. This is understandable in that as the slope increases, the detected positive signal is higher, or closer to an expected positive signal, and it is therefore easier to accurately detect a positive signal, so that FN's are decreased. Similarly there is a clear relationship between FNR and modal SD: as modal SD increases, FNR increases. This is again understandable in that an increase in the deviation of signals that should all have a normal ratio (e.g., 1) indicates an increase in overall noise and/or variation, thus positive results tend to be hidden in the noise and false negative detections increase.

The relationship between FPR and either feature is more modest and in the case of slope appears to be in the opposite direction to the relationship with FNR. While the different behaviors of FNR and FPR, e.g. as shown above, were initially unexpected, further analysis according to the invention has shown that, by the nature of the p-value and SA algorithms in example reader software, FPR should in principle be independent of quality, and determined only by the chosen value of alpha. In practice however, FPR does vary a little, and generally FPR appears to be somewhat inversely correlated with FNR. This is believed to be an artifact of the detection methods employed that causes the calibration of p-values against the chosen alpha level to vary a little from sample to sample. Any such variation that tends to cause an increase in the FNR will simultaneously tend to result in a decrease in FPR, and vice versa. However, it will help in understanding some aspects of the invention to remember that FNR and FPR are not conceptually inverses of each other. FNR is a measure of how “hidden” real signals are, either because the signal strength is weak for some reason or because the background noise or other variance is large. FPR is a measure of how good the detection is in rejecting positive signals that may be caused by spikes in the signal or other variations that are not actually caused by positive signal.

The GenoSensor Reader Software for CGH microarray analysis measures several other quality-associated feature values, as described in the following table.

| | |
|---|---|
| Average spot | The average intrinsic fluorescence intensity of the |
| intensity | spots. This is expressed as the average CCD camera |
| | signal (or “count”) at a pixel, and is corrected |
| | for exposure time. It is intended to represent the |
| | underlying hybridization intensity rather than the |
| | brightness of the captured image, though it will be |
| | affected by the brightness of the lamp. |
| Signal to | The average brightness of spots after the background |
| background | has been subtracted, compared with the average |
| ratio | brightness of the background itself. |
| Median | Ratios of a pair of genomically-adjacent target |
| adjacent-clone | clones should only be different if a breakpoint |
| ratio difference | associated with a copy number change lies between |
| | them. The number of breakpoints is expected always |
| | to be many fewer than the number of target clones |
| | (˜300). Therefore, it is expected that adjacent |
| | clone pairs should have similar ratios in the vast |
| | majority of cases, and the distribution of these |
| | differences will largely be determined by the |
| | “noise” in the system. By finding the median |
| | of the absolute ratio difference between adjacent |
| | clones, we minimize the impact of any breakpoints |
| | associated with a copy number change that may be |
| | present. This measurement should be small; a large |
| | value is indicative of poor quality hybridization. |
| Mean intra- | The average coefficient of variation (standard |
| target CV | deviation/mean) of the replicate spot ratios of |
| | a target. |
| Mean within- | Within any one spot, the per-pixel intensities of |
| spot T/R | the test and reference signals should be very highly |
| correlation | correlated. This measure is the average of the per- |
| | spot correlation coefficients. |
| Modal | The GenoSensor Reader Software identifies a set |
| distribution SD | of of “plausibly modal” targets as part of |
| | the computation of p-values. This is the standard |
| | deviation of the distribution fitted to this set. |
| | It turns out that this measure is strongly |
| | correlated (r = 0.94) with median adjacent- |
| | clone ratio difference. |
| Slope | The parameter that relates observed to expected |
| | ratios. Computed by the SA algorithm. Generally, |
| | higher quality samples have higher slopes. |

Continuous Error Functions

Initial investigation of FNR and FPR were defined at specific (and different) alpha levels, e.g. as used in the scatter plots above showing the correlations with the slope and modal SD quality features. However, because each is based on the thresholding of a finite number of significance values, neither FNR nor FPR is a continuous function of the alpha level. According to specific embodiments of the invention, an alternative formulation avoids this problem:
Eₚₒₛ is the mean of the logarithms of the p-values for ground-truth positive clones (i.e., Eₚₒₛ=mean (log (p)|target ground-truth+ve)). Epos always takes a negative value; more negative values of Eₚₒₛ imply better quality and imply easier detection of positive targets and therefore fewer false negatives. Eₚₒₛ is therefore a continuous-valued analog of FNR.
Similarly, E_{neg} is the mean of the logarithms of the p-values for ground-truth negative clones (i.e., E_{neg}=mean (log(p)|target ground-truth−ve)). E_{neg} always takes a negative value; less negative values of E_{neg} imply better quality and imply easier detection of negative targets and therefore fewer false positives. E_{neg} is therefore a continuous-valued analog of FPR.

The logarithm is used according to specific embodiments of the invention because for a true positive clone, p<0.0001 cannot be considered to be ten times “better” than p<0.001, and certainly p<0.00001 should not be regarded as 100 times better. By using logarithms, p<0.0001 can be regarded as “somewhat better” than p<0.001, and p<0.00001 is still better, but not a lot more so.

The p-values for individual targets are available directly from the p-value analysis method. The Segmental Aneusomy (SA) method as described above computes the p-values of entire segments of target clones that share the same copy number imbalance. For the purposes of computing Eₚₒₛ and E_{neg} when using SA, a suitable p-value can be constructed for each target by considering the SA likelihood function and corresponding p-value for a notional segment comprising just the isolated target; this is referred to herein as the “isolated target p-value”.

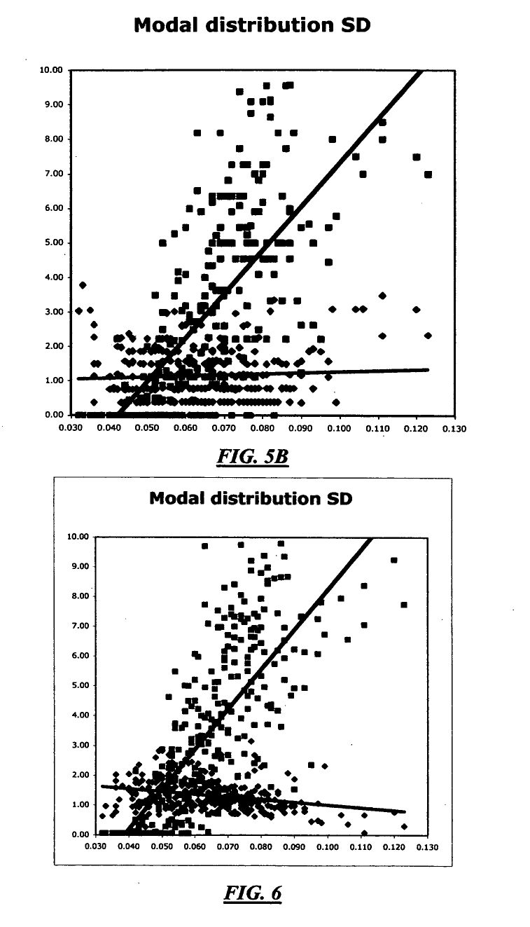

FIG. 6 is an example scatter plot showing Epos (pink) and Eneg (blue) plotted against the same modal SD quality feature as illustrated in FIG. 5 above for FNR and FPR. The much tighter scatter clearly shows the benefit of using continuous error measures. (These and subsequent scatter plots are intended to show correlation between FNR, FPR, Eₚₒₛ, or E_{neg} and a particular quality feature. The values of FNR, FPR, Eₚₒₛ, and E_{neg} have been arbitrarily rescaled to occupy the range 0-10.)

An important advantage to this approach is that it does not rely on correctly guessing or estimating alpha levels; there are no “magic numbers” in the definitions of Eₚₒₛ and E_{neg>}. The reliance on arbitrary choices of alpha levels has been eliminated. In some prior methods, FPR and FNR were determined at specific alpha levels that were chosen generally using ad hoc methods.

Correlations Between Quality Features and the Quality Measures Epos, Eneg

Data for some experimental development were extracted from several hundred captured microarray chip images for which ground truth (or control data) was available. The set included samples of various trisomy cell lines vs. sex-mismatched normal hybridizations; samples of sex-mismatched normal vs. normal hybridizations; samples of microdeletion cell lines vs. sex-mismatched normal hybridizations; and samples of trisomy cell lines vs. sex-mismatched microdeletion cell lines. These microarrays came from a wide variety of batches, and included many “failures”, and so the collection of samples covered a quality continuum that ranged from very good to very poor.

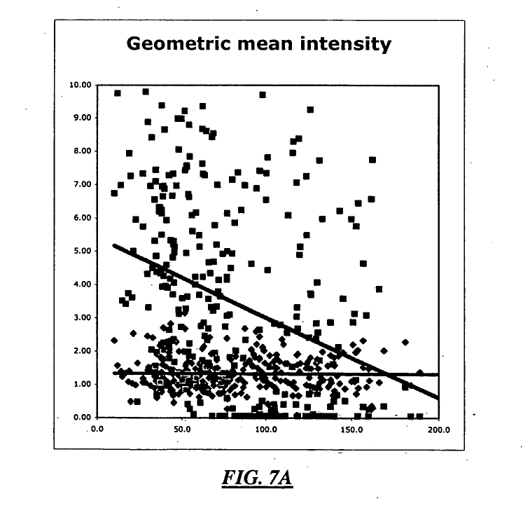

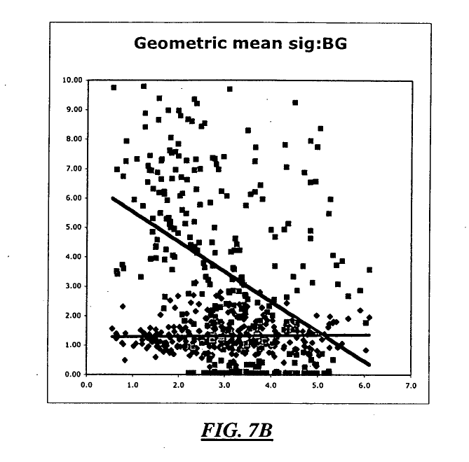

FIG. 7A-B are example scatter plots showing that Epos declines with (A) both increasing Geometric Mean Intensity and (B) increasing Geometric Mean Signal To Background Ratio (sig:BG), which could be a result of increased intensity. These features are mostly familiar from the Quality Measures annotation pane in the software discussed elsewhere herein, except that in the cases of intensity (counts per second) and signal to background ratio the average (geometric mean) of the test and reference values is taken. The relationships of Eₚₒₛ and E_{neg} with slope and with modal SD have already been illustrated and described above.

FIG. 8 is an example scatter plot showing that the Median Adjacent Clone Ratio Difference behaves very similarly to modal distribution SD. This is a nice result because this feature does not depend on the identification of likely modal targets; it therefore can be employed in analysis of cancer chips as well.

As might be expected, the number of missing or excluded spots has been found to generally have little impact on Eₚₒₛ, though it is of course related to the independent quality measure NIR.

“CV of reference intensity” is a novel quality feature that measures the variability of intensity among the target clones on the chip. FIG. 9 is an example scatter plot showing that Epos declines as the variability of target clone intensity (CV) increases.

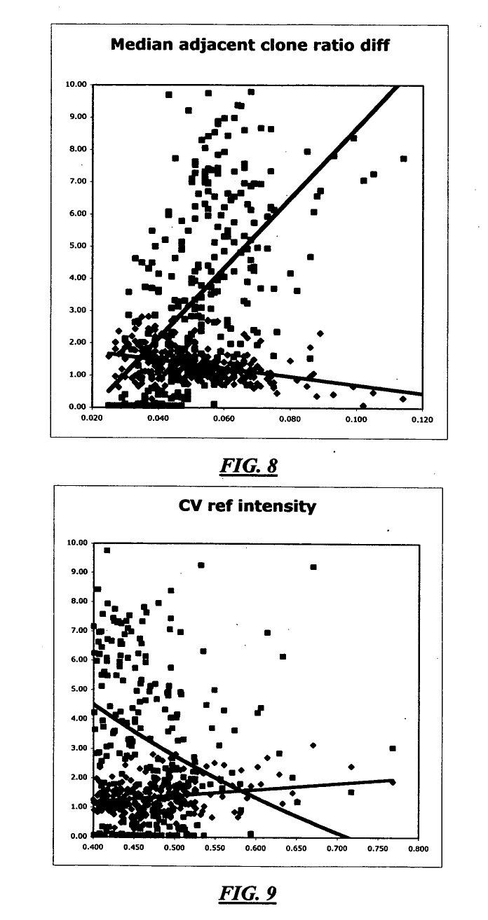

The proportion of saturated plus outlier pixels is also correlated with Eₚₒₛ, as shown in FIG. 10. While this correlation appears rather weak, it is in the opposite direction to what one might expect: a larger proportion of “bad” pixels is associated with a lower Eₚₒₛ.

Definition of Objective Quality Measure

It can be seen that there is generally very little connection between E_{neg} and any of the features. This can be explained as follows. As was explained above, although a lower value of the slope quality feature will likely cause an increased number of false negatives, the value of slope is not expected to have any connection with the occurrence of false positives. In the case of noise quality features such as modal SD or median adjacent clone ratio difference, it might be expected that targets with an observed ratio substantially different to 1.0 on account of a higher overall level of ratio noise would be detected as false positives, leading to an increased number of false positives in the case of noisier samples. This does not occur in practice, because a general reduction in the likelihood values of ratio changes caused by the increased noise level almost completely compensates the general increase in ratio changes. Therefore, increasing values of the noise features should cause an increase in false negatives but have no impact on the number of false positives.

However, it can be seen in some of the panels above that E_{neg} consistently shows a small inverse correlation with Eₚₒₛ. The cause of this is believed to be small errors in estimation of internal parameters of the Segmental Aneusomy algorithm. In particular, small errors in estimation of the variances vᵢ would not be surprising. Their effect would be to add a consistent bias to both likelihood and significance values, which in turn would be equivalent to a small change in the p-value threshold (or alpha). Over a set of samples, such random small changes in the effective value of the p-value threshold would explain the observed correlation.

This small inverse correlation of E_{neg} with Eₚₒₛ provides a reason to include a balanced combination of E_{neg} and Eₚₒₛ in the final definition of quality. These data and considerations lead to the proposal that the overall measure of quality of a microarray analysis is well represented by the error function E_{neg}−Eₚₒₛ, known as the “overall quality rating” or OQR. E_{neg}-Eₚₒₛ may take either positive or negative value depending on the overall quality; larger positive values of OQR imply a higher quality microarrays.

Predicting an Objective “Overall Quality Rating” (OOR) By Multiple Regression

The quality feature data from a set of chip images taken together with ground-truth values of the overall quality rating OQR can be used as a training set to develop an algorithm to predict the value of OQR in the case of novel samples with unknown ground truth. Ideally, the algorithm should not just separate samples into the two categories “good” and “bad”, but should estimate a continuous value of OQR. If a two-class solution is required, this can then be obtained by applying a threshold to the estimated value of OQR.

Because Eₚₒₛ and E_{neg} show correlation to varying degrees with a number of the quality features, multiple regression was used to develop a “model” that predicts the value of OQR in unknown samples. Conventional multiple regression models a dependent variable (OQR) as a linear function of independent variables (the quality feature values). By applying appropriate transformations to the quality feature data, arbitrary multiple regression functions (e.g. polynomial, logarithmic) can be constructed, and some of these options have been investigated.

The results presented here are based on 4-parameter multiple linear regression models. The parameters selected in this example are: (1) sqrt(slope), (2) log(median adjacent clone ratio difference), (3) log(reference intensity CV), (4) square(geometric mean signal to background).

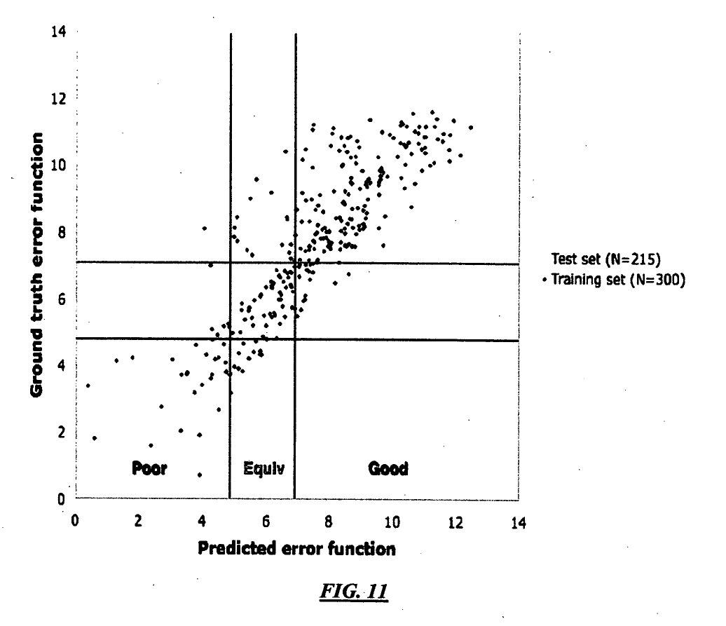

The results are shown as a scatter plot between the ground-truth value of OQR (Y-axis), which is based on the known copy number changes in the DNAs used to produce the data set, and the predicted value of OQR (X-axis), calculated as a linear combination of the chosen features. (Note that OQR as defined sometimes has a negative value. The scatter plot in FIG. 11 shows the value used in practice, OQR′=OQR+k, where k is chosen so that OQR′ is always positive, with very poor samples obtaining a value close to zero.) Blue spots are from 300 mixed-quality samples used to train the multiple regression model, while yellow spots are from an independent test set of 215 mixed-quality samples that were not used for model training.

The horizontal pink and red lines at the median and 20^{th} percentile respectively of the ground-truth OQR′ values of the training data divide the training data into three sets, which can be thought of as ground truth “good”, “equivocal” and “poor” quality. The vertical pink and red lines have the same OQR′ values; these lines can be used to classify unknown samples as “good”, “equivocal” or “poor” based on their predicted value of OQR′. Samples lying outside the three square regions along the diagonal are misclassified. It can be seen that just one ground-truth “good” sample has been classified as “poor”, while no “poor” sample has been classified as “good”. While a number of samples have been less seriously misclassified, e.g. “good” samples classified as “equivocal”, the great majority have been given the correct OQR′ class.

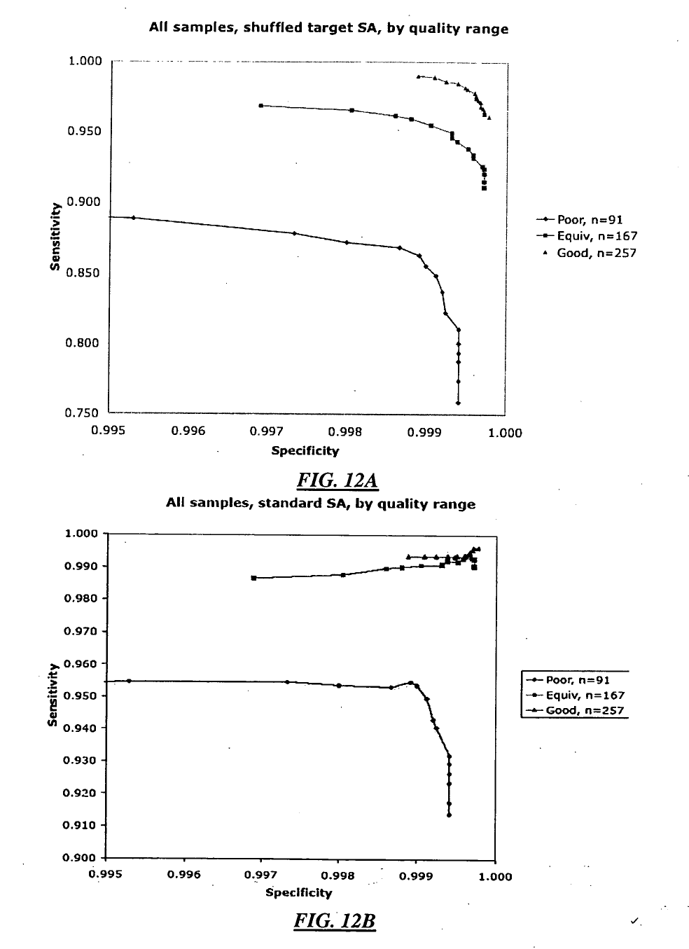

The impact of the quality classes on SA performance is shown by the receiver operating characteristic (ROC) curves illustrated in FIG. 12A&B, where the data set has been triaged into the three quality classes by the predicted value of OQR. It can be seen that OQR is very successful in identifying those samples that go on to have the poorest performance. FIG. 12B shows analytical sensitivity and specificity (ROC curves) for 515 sex-mismatched hybridizations [developmental array with 287 clones], comprising 129 normal donor blood specimens and 386 cell line samples. It is evident that different sample qualities result in radically different ROCs, with markedly improved sensitivity and specificity in higher-quality samples. A significance level can be chosen from the ROC curve. In this example, it was chosen as P<0.0001 for SA algorithm, and P<0.001 for the old, Non Modal P value method calculation algorithm (not shown).

Discussion

The data presented show that, as expected, FNR varies widely among chips, from near-zero to near-100%. FPR is, as expected, largely determined by the alpha level. Therefore, the most obvious objective outcome of differences in-chip preparation quality will be differences in the FNR or its continuous analog Eₚₒₛ. But FPR does nevertheless show inverse correlation with FNR to a small degree (and Eₚₒₛ with E_{neg}). This can be explained as a consequence of small errors in estimating internal parameters of the SA algorithm, which has the effect of moving the operating point along the ROC curve. This small correlation provides a reason for also including E_{neg} in the objective definition of the overall chip analysis quality rating OQR.

An objective quality measure with practical utility according to specific embodiments of the invention uses a suitable combination of false negative and false positive rates or their continuous analogs Eₚₒₛ and E_{neg}. If such a quality measure is estimated for an analysis where the ground truth is unknown, it then predicts the relative frequency of target errors in the analysis. In short, a sample with a higher value of such a measure (as defined here) will likely have more FNs and/or FPs. Such a measure can therefore be used to advise the user how much reliance can be placed in the results; or it can be used to reject a sample entirely. It may also be used to triage results into three classes: (i) accept results without further confirmation; (ii) confirm all positive results with an additional test; or (iii) reject the sample.

Data presented here show that FNR, whether measured at a particular alpha level or by Eₚₒₛ, the average logarithm of the p-value of positive target clones, is very strongly correlated with a number of quality features that can be measured from the chip image without prior knowledge of the ground truth. FPR and E_{neg} also show a degree of correlation with some of the features, though to a lesser extent.

The results also show that an overall quality rating defined as a weighted sum of FNR and FPR or their analogs can be estimated from the quality feature values. Comparing the estimated OQR value against a threshold or thresholds can be used to decide whether to accept or reject a microarray analysis on the grounds of quality, i.e., provides a quality control.

How to set an appropriate threshold or thresholds for actual use will vary in different embodiments and can be dependent on the formal requirements of particular systems. Here it has been proposed to use to two thresholds, to divide the quality range into classes “good”, “equivocal” and “poor”. Almost no samples are misclassified between the “good” and “poor” quality classes.

In some situations, the optimum regression parameters may need to be changed as the evolution of the assay changes the distribution of feature values and/or the correlations between feature values and performance. It would be wise to continue to collect additional data for quality measure training on an ongoing basis.

The regression analysis itself may be further optimized, for example by investigating other possible combinations of features or of feature transformations such as log(.) and exp(.).

An objective quality measure (error function) for use with either the SA or the p-value method can be defined as OQR=E_{neg}−Eₚₒₛ. Because the positive and negative targets are not known, its value according to embodiments of the invention as described above is estimated by a linear function of quality feature values (where, in various embodiments, these quality feature values may be transformed by such functions as square, exp, or log). The linear function parameters can be trained by multiple regression analysis of suitable training data known to incorporate both good and bad chips, but without requiring any subjective classification of the individual chips into “good” and “bad” classes.

A second quality measure is the proportion of non-informative target clones (NIR). Since this can be measured directly by the analysis software, it can be used separately. Each such of these measures could be used in combination with a threshold, to divide analyses into two classes “accept” and “reject”. Given such thresholds, the proportion of rejected chips in a given population will be largely determined by the quality of the assay across the population. Alternatively, a more detailed categorization could be applied, e.g. into three classes “accept”, “accept after verification”, “reject”. Or the quality measure value could simply be presented to the user together with advice on its likely consequences.

Thus, in specific embodiments, as described above, the present invention can be incorporated into one or more logic modules or components for an in vitro diagnostic system, such as the GenoSensor Reader Software. In various embodiments, a diagnostic system can include logic instructions and/or modules for one or more of:
Computing the overall quality rating (OQR) value for a chip. Specification of which quality features should be used, their preliminary transformations, and the linear function parameters may all be encoded in a parameters file.
Prominently presenting both the OQR and the non-informative rate to the user.
Applying thresholds specified in the parameters file in order to classify the sample as “accept” or “reject”, and requiring such outcome to be present on the final Report printed by the analysis software.

In further embodiments, chip image data should continue to be collected for training and verifying the quality measure estimation, in order to track subtle long-term changes in the assay. Whenever there is a step change in the assay, entirely replacing the quality training set should be considered.

In further embodiments, feature selection, feature transformations, and the linear function, can be adapted and optimized for the SA method.

Other Diagnostic Uses

As described above, following identification and validation of a particular assay producing observable data sets and training statistical analysis parameters and selecting quality features as describe above, assay analysis methods according to specific embodiments of the invention can be used in clinical or research settings, such as to predictively categorize subjects into disease-relevant classes, to monitor subjects for developmental disregulations, etc. Systems and/or methods of the invention can be utilized for a variety of purposes by researchers, physicians, healthcare workers, hospitals, laboratories, patients, companies and other institutions. For example, the invention can be applied to: diagnose disease; assess severity of disease; predict future occurrence of disease; predict future complications of disease; determine disease prognosis; evaluate the patient's risk; assess response to current drug therapy; assess response to current non-pharmacologic therapy; determine the most appropriate medication or treatment for the patient; and determine most appropriate additional diagnostic testing for the patient, among other clinically and epidemiologically relevant applications. Essentially any disease, condition, or status for which an assay producing statistically analyzable data exists or can be developed can be more reliably detected using the diagnostic methods of the invention, see, e.g. Table 2.

In addition to assessing health status at an individual level, the methods and diagnostic sensors of the present invention are suitable for evaluating subjects at a “population level,” e.g., for epidemiological studies, or for population screening for a condition or disease.

Web Site Embodiment

The methods of this invention can be implemented in a localized or distributed data environment. For example, in one embodiment featuring a localized computing environment, an assay reader according to specific embodiments of the present invention is configured in proximity to a desired diagnostic area, which is, in turn, linked to a computational device equipped with user input and output features. In a distributed environment, the methods can be implemented on a single computer, a computer with multiple processes or, alternatively, on multiple computers.

Kits

A diagnostic assay according to specific embodiments of the present invention is optionally provided to a user as a kit. Typically, a kit of the invention contains one or more genetic targets constructed according to the methods described herein. Most often, the kit contains one or more DNA targets packaged or affixed in a suitable container. The kit optionally further comprises an instruction set or user manual detailing preferred methods of using the kit components for performing an assay of interest.

When used according to the instructions, the kit enables the user to identify diseases or conditions using patient tissues, including, but not limited to cellular interstitial fluids, whole blood, amniotic fluid, supernatant, etc. The kit can also allow the user to access a central database server that receives and provides information to the user and that may perform data analysis and or assay quality analysis. Additionally, or alternatively, the kit allows the user, e.g., a health care practitioner, clinical laboratory, or researcher, to determine the probability that an individual belongs to a clinically relevant class of subjects (diagnostic or otherwise).

Embodiment in a Programmed Information Appliance

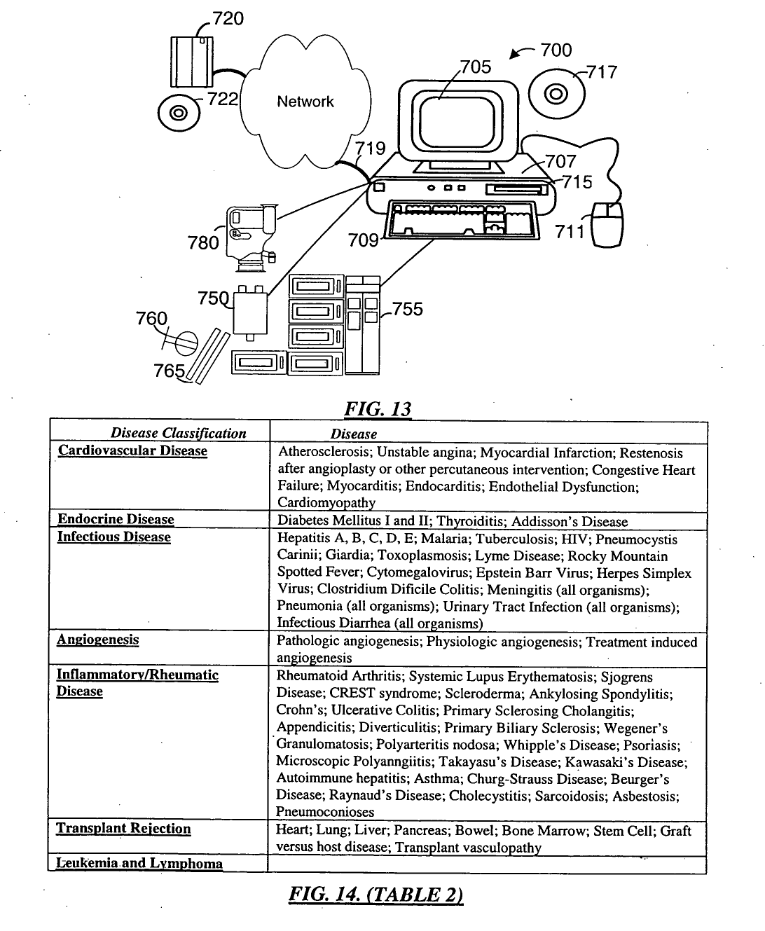

FIG. 13 is a block diagram showing a representative example logic device and/or diagnostic system in which various aspects of the present invention may be embodied. As will be understood from the teachings provided herein, the invention can be implemented in hardware and/or software. In some embodiments, different aspects of the invention can be implemented in either client-side logic or server-side logic. Moreover, the invention or components thereof may be embodied in a fixed media program component containing logic instructions and/or data that when loaded into an appropriately configured computing device cause that device to perform according to the invention. A fixed media containing logic instructions may be delivered to a viewer on a fixed media for physically loading into a viewer's computer or a fixed media containing logic instructions may reside on a remote server that a viewer accesses through a communication medium in order to download a program component.

FIG. 13 shows an information appliance or digital device 700 that may be understood as a logical apparatus that can perform logical operations regarding image display and/or analysis as described herein. Such a device can be embodied as a general purpose computer system or workstation running logical instructions to perform according to specific embodiments of the present invention. Such a device can also be custom and/or specialized laboratory or scientific hardware that integrates logic processing into a machine for performing various sample handling operations. In general, the logic processing components of a device according to specific embodiments of the present invention is able to read instructions from media 717 and/or network port 719, which can optionally be connected to server 720 having fixed media 722. Apparatus 700 can thereafter use those instructions to direct actions or perform analysis as understood in the art and described herein. One type of logical apparatus that may embody the invention is a computer system as illustrated in 700, containing CPU 707, optional input devices 709 and 711, storage media (such as disk drives) 715 and optional monitor 705. Fixed media 717, or fixed media 722 over port 719, may be used to program such a system and may represent a disk-type optical or magnetic media, magnetic tape, solid state dynamic or static memory, etc. The invention may also be embodied in whole or in part as software recorded on this fixed media. Communication port 719 may also be used to initially receive instructions that are used to program such a system and may represent any type of communication connection.

FIG. 13 shows additional components that can be part of a diagnostic system in some-embodiments. These components include a viewer 750, automated slide or microarray stage 755, light (UV, white, or other) source 760 and optional filters 765, and a CCD camera or capture device 780 for capturing digital images for analysis as described herein. It will be understood to those of skill in the art that these additional components can be components of a single system that includes logic analysis and/or control. These devices also may be essentially stand-alone devices that are in digital communication with an information appliance such as 700 via a network, bus, wireless communication, etc., as will be understood in the art. It will be understood that components of such a system can have any convenient physical configuration and/or appear and can all be combined into a single integrated system. Thus, the individual components shown in FIG. 13 represent just one example system.

The invention also may be embodied in whole or in part within the circuitry of an application specific integrated circuit (ASIC) or a programmable logic device (PLD). In such a case, the invention may be embodied in a computer understandable descriptor language, which may be used to create an ASIC, or PLD that operates as herein described.

Other Embodiments

The invention has now been described with reference to specific embodiments. Other embodiments will be apparent to those of skill in the art. In particular, a viewer digital information appliance has generally been illustrated as a personal computer. However, the digital computing device is meant to be any information appliance suitable for performing the logic methods of the invention, and could include such devices as a digitally enabled laboratory systems or equipment, digitally enabled television, cell phone, personal digital assistant, etc. Modification within the spirit of the invention will be apparent to those skilled in the art. In addition, various different actions can be used to effect interactions with a system according to specific embodiments of the present invention. For example, a voice command may be spoken by an operator, a key may be depressed by an operator, a button on a client-side scientific device may be depressed by an operator, or selection using any pointing device may be effected by the user.

It is understood that the examples and embodiments described herein are for illustrative purposes and that various modifications or changes in light thereof will be suggested by the teachings herein to persons skilled in the art and are to be included within the spirit and purview of this application and scope of the claims.

All publications, patents, and patent applications cited herein or filed with this application, including any references filed as part of an Information Disclosure Statement, are incorporated by reference in their entirety.

What is claimed is:

1. A method of determining and reporting a diagnostic assay result using a computer system comprising:
receiving observed data captured from one or more observable targets of said diagnostic assay at said computer system;
using a portion of said observed data to determine one or more assay results;
determining two or more quality features of said diagnostic assay from said observed data;
using said two or more quality features to predict an error function;
using said error function to determine and report a quality measure for said diagnostic assay;
using said quality measure in making a final report of said assay result.

2. The method according to claim 1 further wherein said error function is predicted using a statistical model, said statistical model having one or more parameters derived from one or more training assays.

3. The method according to claim 1 further wherein said error function is predicted using a statistical model, said statistical model having one or more parameters trained using known ground truth samples and their corresponding diagnostic assay results.

4. The method according to claim 1 wherein said diagnostic assay result indicates the presence or absence of one or more DNA sequence copy number changes indicative of cancerous or precancerous cells.

5. The method according to claim 1 wherein said diagnostic assay result indicates the presence or absence of one or more DNA sequence copy number changes indicative of one or more congenital abnormalities.

6. The method according to claim 1 further comprising:
wherein said determining two or more quality features uses observed data of two or more of a group of said targets; and
wherein said error function is predicted for multiple targets of said group.

7. The method according to claim 6 further comprising:
wherein said group comprises a plurality of targets on a genomic analysis chip; and
wherein said error function is predicted for all or nearly all targets on said chip.

8. The method according to claim 7 further wherein:
said chip has more than about 50 separable targets;
each said separable target is an assay; and
each of said assays is either positive or negative for altered DNA copy number.

9. The method according to claim 1 wherein said observed data is captured from performing said assay on a test sample preparation comprising one or more of:
a portion of a tissue biopsy;
a cellular monolayer prepared from disaggregated cells;
a cellular suspension in a fluid or a gel;
a smear preparation; or
cellular derived material.

10. The method according to claim 1 further comprising:
selecting from available quality features those that are associated in some way with an error function.

11. The method according to claim 1 further comprising:
selecting from available quality features, features associated with an error function, said features being two or more selected from the group consisting of:
median adjacent-target signal ratio difference;
attenuation of measured to expected signals;
signal to background ratio;
average target signal intensity;
missing/excluded targets;
outlier/saturated target signal detection;
mean intra-target coefficient of variation;
mean within-target test and reference signal correlation;
modal distribution standard deviation.

12. The method according to claim 1 further comprising:
using an estimate of ratio noise as a quality feature to predict an error function.

13. The method according to claim 12 further comprising:
using the median adjacent-target ratio difference to predict an error function.

14. The method according to claim 1 further comprising:
using an estimate of a signal level of positive targets as a quality feature to predict an error function.

15. The method according to claim 14 further comprising:
using an average attenuation from positive control targets as a signal level quality feature to predict an error function.

16. The method according to claim 14 further comprising:
using an average attenuation estimated by a segmental aneusomy algorithm as a signal level quality feature to predict an error function.

17. The method according to claim 1 further wherein:
said observed data comprises a captured image of a microarray of assay targets.

18. The method according to claim 1 further comprising:
expressing said error function as an estimated value of a function of the false positive rate and false negative rate for an assay sample, when true values of said false positive and false negative rates are unknown for the assay.

19. The method according to claim 1 further comprising:
training said error function using measurable features from known control samples data.

20. The method according to claim 19 further comprising:
training said error function from measurable features from known control samples data by building a multiple regression model.

21. The method according to claim 19 further comprising:
training said error function by building a multiple non-linear regression model from known control samples data by applying non-linear transformations to said measurable features.

22. The method according to claim 1 further comprising:
using a difference function E_{neg}-Eₚₒₛ as said error function where Eₚₒₛ is a mean of the logarithms of the p-values for ground-truth positive clones and E_{neg} is a mean of the logarithms of the p-values for ground-truth negative clones.

23. A method to detect copy number change using a DNA microarray and a computer system comprising:
modeling ratio changes that extend across a segment of adjacent targets; and
using a maximum likelihood analysis in said modeling.

24. The method according to claim 23 further comprising:
accepted or not accepted changes according to formal significance criteria based on chi-square.

25. The method according to claim 23 further wherein said maximum likelihood modeling is constrained to model only appropriate ratios.

26. The method according to claim 25 wherein appropriate ratios are determined using a reference DNA with a copy number of 1 or 2 and target DNA copy numbers of 0, 1, 2, 3, or 4.

27. The method according to claim 25 wherein said image is a two-dimensional image.

28. A system for analyzing biologic samples comprising:
an information processor for handling digital data;
data storage for storing digital data, including captured image data;
a logic module able to analyze said captured image data to estimate observable features of said data and able to predict an error rate using selected observable features.

29. The system of claim 28 further comprising:
an image capture camera operationally connected to said information processor;
a light source;
a viewer;
an array handling unit.

30. The system of claim 28 further comprising:
one or more rule sets for predicting error functions stored in said data storage.

31. The system of claim 28 further comprising:
one or more analysis logic routines stored in said data storage.

32. A system for analyzing biologic samples comprising:
means for capturing digital image data from one or more biologic samples;
means for storing digital image data;
means for interacting with a user to receive user instructions and user review of image data; and
means for logically analyzing said captured digital image data to predict one or more error functions from detectable features; and
means for outputting predicted error functions to a user.

33. A method of screening for congenital genetic abnormalities in a subject using a computer system comprising:
receiving captured data from a set of separable targets, each target providing observable data indicative of genetic sequence copy number at a particular chromosomal location;
analyzing said captured data using a segmental aneusomy statistical analysis method that groups targets into segments indicating adjacent chromosomal regions, each segment representing a region having a same copy number imbalance;
thereby from one assay detecting both segmental and whole chromosome changes in copy number.

34. The method according to claim 33 further comprising:
modeling ratio changes that extend across a segment of adjacent targets; and
using a maximum likelihood analysis in said modeling.

35. The method according to claim 34 further comprising:
accepted or not accepted changes according to formal significance criteria based on chi-square.

36. The method according to claim 34 further wherein said maximum likelihood modeling is constrained to model only appropriate ratios.

37. The method according to claim 36 wherein appropriate ratios are determined using a reference DNA with a copy number of 1 or 2 and target DNA copy numbers of 0, 1, 2, 3, or 4.

38. The method according to claim 33 further comprising:
providing a comparative genomic hybridization array of multiple targets for a genome, wherein telomeres and chromosomal regions associated with known microdeletions/microduplications of interest are represented by two or more closely spaced target sequences on the array;
hybridizing a test sample from a subject to said array; and
capturing an image of said array.

39. The method according to claim 38 further wherein said array and said statistical method are optimized to detect chromosomal imbalances that are a common cause of developmental disorders such as mental retardation/developmental delay, physical birth defects and dysmorphic features.

40. The method according to claim 33 further comprising:
from one assay detecting whole chromosome aneusomies, microdeletions, microduplications and unbalanced subtelomeric (subTel) rearrangements.

41. The method according to claim 33 further wherein said subject is selected from the group comprising:
a prenatal mammal fetus;
a pre-implantation mammalian embryo; and
a postnatal mammal.

42. The method according to claim 41 further wherein a whole-chromosomal sample is extracted without harm to said subject.

43. The method according to claim 41 further wherein said subject is human.

44. The method according to claim 33 further wherein:
said assay does not require reciprocal hybridizations; and
said assay reliably detects copy number abnormalities (CNAs) from both fresh and fixed peripheral blood or cell line specimens.

45. The method according to claim 33 further wherein:
said method is incorporated into a system that:
automates hybridization and washing;
automates image capture and data analysis;
assesses the quality of the assay; and
reports qualitative results (gain, loss, no change); and
further wherein software associated with said system controls image acquisition, analysis, and data reporting.

46. The method according to claim 45 further wherein:
said software identifies spots based on the DAPI signal, measures mean intensities from the green and red image planes, subtracts background, determines the ratio of green/red signal, and calculates the ratio most representative of the modal DNA copy number of the sample DNA.

47. The method according to claim 33 further comprising:
providing an array of target clones wherein clones of are identified and further at a minimum 3 clones are chosen per chromosome arm, with at least 82 subtelomeric clones and 29 clones in known microdeletion/microduplication regions;
and further wherein each telomere, other than the acrocentric chromosome p arms, is represented by two clones.
and further wherein each microdeletion/microduplication region is represented by 2 to 5 clones.

48. A computer readable medium containing computer interpretable instructions that when loaded into an appropriately configuration information processing device will cause the device to operate in accordance with the method of claim 1.

49. A computer readable medium containing computer interpretable instructions that when loaded into an appropriately configuration information processing device will cause the device to operate in accordance with the method of claim 23.
